(19) United States
(12) Patent Application Publication
(10) Pub. No.: US 2024/0339228 A1
(43) Pub. Date: Oct. 10, 2024

(54) SYSTEM, METHOD, AND COMPUTER PROGRAM FOR PROVIDING COGNITIVE AND SOCIAL THERAPY SERVICES TO CHILDREN WITH DEVELOPMENTAL DISABILITIES

(71) Applicants: DUBU INC., Seoul (KR); UIF (UNIVERSITY INDUSTRY FOUNDATION), YONSEI UNIVERSITY, Seoul (KR); RUDOLPH CHILD RESERCH CENTER INC., Seoul (KR)

(72) Inventors: Hye Jin KAM, Seongnam-si Gyeonggi-do (KR); Kang Hyuk DO, Incheon (KR); Yu Rang PARK, Seoul (KR); Yun Joo KOH, Suncheon-si Jeollanam-do (KR)

(73) Assignees: DUBU INC., Seoul (KR); UIF (UNIVERSITY INDUSTRY FOUNDATION), YONSEI UNIVERSITY, Seoul (KR); RUDOLPH CHILD RESERCH CENTER INC., Seoul (KR)

(21) Appl. No.: 18/737,359

(22) Filed: Jun. 7, 2024

Related U.S. Application Data
(63) Continuation of application No. PCT/KR2023/011480, filed on Aug. 4, 2023.

(30) Foreign Application Priority Data
Apr. 7, 2023 (KR) 10-2023-0046342

(51) Int. Cl. G16H 80/00; A61B 5/00; A61B 5/16; G16H 10/60; G16H 15/00; G16H 50/30; G16H 50/70

(52) U.S. Cl. CPC G16H 80/00; A61B 5/165; A61B 5/744; G16H 10/60; G16H 15/00; G16H 50/30; G16H 50/70

(57) ABSTRACT

A treatment service mediation server includes a metaverse space providing unit configured to provide a metaverse space for the cognitive and social treatment service to a device of the child with developmental disabilities and a device of a therapist; a data collection unit configured to collect life-logging data including user data and cognitive and social response data of the child with developmental disabilities; a mental state estimation unit configured to estimate a mental state of the child with developmental disabilities from the life-logging data through an artificial intelligence model; a treatment service providing unit configured to provide the cognitive and social treatment service of the child with developmental disabilities to the device of the child with developmental disabilities based on the estimated mental state of the child with developmental disabilities; and a treatment result providing unit configured to provide a treatment result report on the cognitive and social treatment service.

### CROSS-REFERENCE TO RELATED APPLICATIONS

This application is a continuation of International Application of PCT/KR2023/011480 filed on Aug. 4, 2023, which claims priority to Korean Patent Application No. 10-2023-0046342 filed on Apr. 7, 2023, the entire contents of which are herein incorporated by reference.

### TECHNICAL FIELD

The present disclosure relates to a server, method and computer program for providing a cognitive and social treatment service of a child with developmental disabilities.

### BACKGROUND

Autism spectrum disorder (ASD) is a disorder characterized by deficits in social interaction, verbal and nonverbal communication and social behavior.

Children with ASD are under a lot of stress about the development of language and the maintenance of relation and cannot learn social behaviors due to deficits in interactions with others and weak wills for interactions. Thus, most of developmental areas, such as language, cognitive and learning skills, of the children with ASD are negatively affected.

In particular, children without intellectual disability (i.e., children with an IQ of 70 or higher) among children diagnosed with ASD are considered as having high functioning ASD (HF-ASD) according to DSM-5 diagnostic criteria (Diagnostic and Statistical Manual of Mental Disorders).

Children with HF-ASD may have IQs and academic achievement levels similar to or higher than those of normal children, but have difficulty in reading the minds of others and low understanding in verbal communication and nonverbal signals regardless of intellectual and language abilities. Thus, the children with HF-ASD show an increased risk of bullying victimization. Also, the children with HF-ASD may have depression, anxiety or obsessive-compulsive or tic disorders due to lack of social skills, and in severe cases, they may have schizophrenia.

Meanwhile, Korea has a shortage of specialized treatment centers and specialists in charge of treating children with HF-ASD, and, thus, the children with HF-ASD have difficulty in receiving treatment and need to wait for a long time to use treatment.

Existing specialized treatment centers train children with HF-ASD only for some social situations for safety reasons and thus are limited in providing various scenarios that can be encountered in real life.

Even if children with HF-ASD participate in a treatment program, the price of the program is considerable and additional treatment costs can be incurred, which leads to a heavy financial burden on their parents.

### DISCLOSURE OF THE INVENTION

### Problems to be Solved by the Invention

The present disclosure is to solve the above problems, and provided to estimate a mental state of a child with developmental disabilities during a diagnostic process in a metaverse space from life-logging data of the child with developmental disabilities and to provide a cognitive and social treatment service to a device of the child with developmental disabilities based on the estimated mental state of the child with developmental disabilities.

However, the problems to be solved by the present disclosure are not limited to the above-described problems. There may be other problems to be solved by the present disclosure.

### Means for Solving the Problems

According to an aspect of the present disclosure, a treatment service mediation server that provides a cognitive and social treatment service of a child with developmental disabilities includes a metaverse space providing unit configured to provide a metaverse space for the cognitive and social treatment service to a device of the child with developmental disabilities and a device of a therapist; a data collection unit configured to collect life-logging data including user data and cognitive and social response data of the child with developmental disabilities during a diagnostic process in the metaverse space; a mental state estimation unit configured to utilize an artificial intelligence model to estimate a mental state of the child with developmental disabilities based on the collected life-logging data; a treatment service providing unit configured to provide the cognitive and social treatment service of the child with developmental disabilities to the device of the child based on the estimated mental state; and a treatment result providing unit configured to provide a treatment result report on the cognitive and social treatment service to a device of parents of the child.

According to another aspect of the present disclosure, a treatment service mediation method that provides a cognitive and social treatment service of a child with developmental disabilities and is performed by a treatment service mediation server includes providing a metaverse space for the cognitive and social treatment service to a device of the child with developmental disabilities and a device of a therapist; collecting life-logging data including user data and cognitive and social response data of the child with developmental disabilities during a diagnostic process in the metaverse space; utilizing an artificial intelligence model to estimate a mental state of the child with developmental disabilities based on the collected life-logging data; providing the cognitive and social treatment service of the child with developmental disabilities to the device of the child based on the estimated mental state of the child; and providing a treatment result report on the cognitive and social treatment service to a device of parents of the child.

According to yet another aspect of the present disclosure, a computer program stored in a computer-readable medium including a sequence of instructions for providing a cognitive and social treatment service of a child with developmental disabilities, which when executed by a computing device, cause a computing device to provide a metaverse space for the cognitive and social treatment service to a device of the child with developmental disabilities and a device of a therapist; collect life-logging data including user data and cognitive and social response data of the child with developmental disabilities during a diagnostic process in the metaverse space; utilize an artificial intelligence model to estimate a mental state of the child with developmental disabilities based on the collected life-logging data; provide the cognitive and social treatment service of the child with developmental disabilities to the device of the child based on the estimated mental state of the child; and provide a treatment result report on the cognitive and social treatment service to a device of parents of the child.

The above-described aspects are provided by way of illustration only and should not be construed as liming the present disclosure. Besides the above-described embodiments, there may be additional embodiments described in the accompanying drawings and the detailed description.

### Effects of the Invention

According to any one of the above-described means for solving the problems of the present disclosure, it is possible to estimate a mental state of a child with developmental disabilities during a diagnostic process in a metaverse space from life-logging data of the child with developmental disabilities and to provide a cognitive and social treatment service to a device of the child with developmental disabilities based on the estimated mental state of the child with developmental disabilities.

Therefore, according to the present disclosure, the child with developmental disabilities can have various learning opportunities of interacting with others in a metaverse space.

Also, according to the present disclosure, non-face-to-face treatment is provided through the metaverse space, and, thus, it is possible to overcome the time and space limitations of conventional treatment and also possible to reduce a financial burden.

### BRIEF DESCRIPTION OF THE DRAWINGS

FIG. 1 is a block diagram of a treatment service mediation server according to an embodiment of the present disclosure.

FIG. 2 is provided to explain a method of collecting life-logging data of a child with developmental disabilities through a metaverse space according to an embodiment of the present disclosure.

FIG. 3 shows topics of non-face-to-face treatment scenarios for treating the child with developmental disabilities according to an embodiment of the present disclosure.

FIG. 4A to FIG. 4F illustrate examples of metaverse-dedicated treatment spaces according to an embodiment of the present disclosure.

FIG. 5 is provided to explain a therapist-dedicated interface according to an embodiment of the present disclosure.

FIG. 6 illustrates an example of a parental metaverse space according to an embodiment of the present disclosure.

FIG. 7A to FIG. 7C are provided to explain a treatment result report on a cognitive and social treatment service according to an embodiment of the present disclosure.

FIG. 8 is a flowchart showing a method of providing a cognitive and social treatment service of the child with developmental disabilities according to an embodiment of the present disclosure.

### DETAILED DESCRIPTION

Hereinafter, embodiments of the present disclosure will be described in detail with reference to the accompanying drawings so that the present disclosure may be readily implemented by a person with ordinary skill in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Additionally, when a part “includes” one element, this means that it may further include another element rather than excluding another element, unless specifically stated to the contrary.

Through the whole document, the term “unit” includes a unit implemented by hardware, a unit implemented by software, and a unit implemented by both of them. One unit may be implemented by two or more pieces of hardware, and two or more units may be implemented by one piece of hardware.

Through the whole document, a part of an operation or function described as being carried out by a terminal or device may be carried out by a server connected to the terminal or device. Likewise, a part of an operation or function described as being carried out by a server may be carried out by a terminal or device connected to the server.

Hereinafter, the present disclosure will be explained in detail with reference to the accompanying configuration views or process flowcharts.

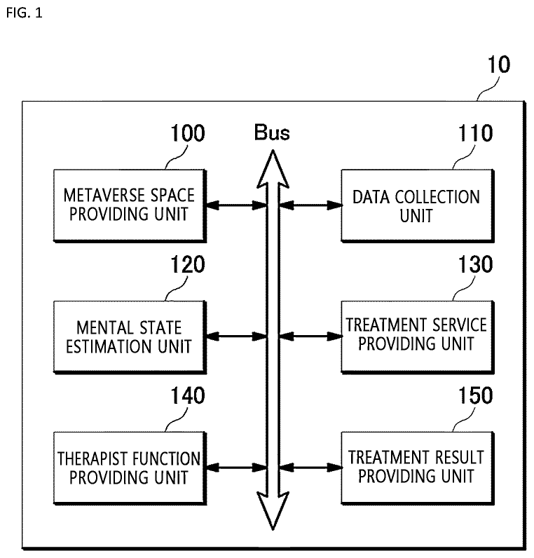

FIG. 1 is a block diagram of a treatment service mediation server 10 according to an embodiment of the present disclosure.

Referring to FIG. 1, the treatment service mediation server 10 may include a metaverse space providing unit 100, a data collection unit 110, a mental state estimation unit 120, a treatment service providing unit 130, a therapist function providing unit 140, and a treatment result providing unit 150. However, the treatment service mediation server 10 illustrated in FIG. 1 is merely an embodiment of the present disclosure, and various modifications based on the components illustrated in FIG. 1 are possible. The treatment service mediation server 10 may include one or more processors and one or more memory modules including programs. The metaverse space providing unit 100, the data collection unit 110, the mental state estimation unit 120, the treatment service providing unit 130, the therapist function providing unit 140, and the treatment result providing unit 150 may be program modules in the form of operating systems, application program modules, and other program modules stored in the memory modules. Such program modules may include, but are not limited to, routines, subroutines, programs, objects, components, and data structures for performing specific tasks or executing specific abstract data types according to the invention as will be described below.

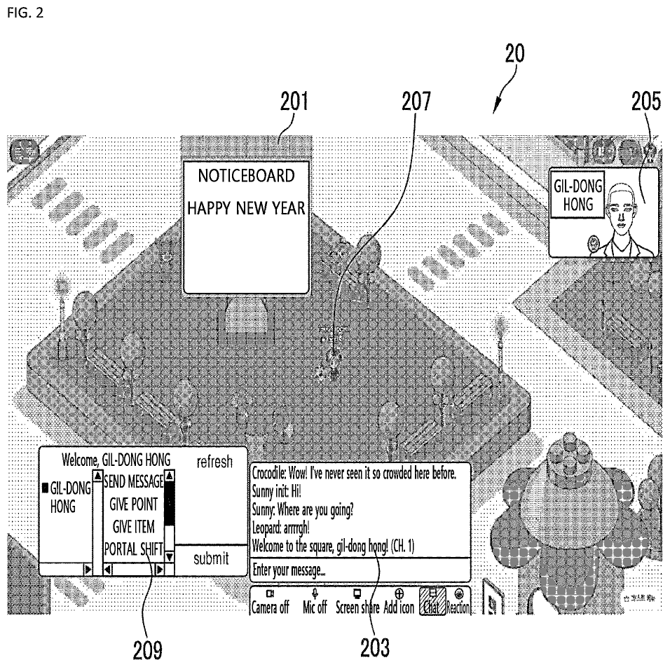

The following description will be made with reference to FIG. 1 as well as FIG. 2 to FIG. 7.

The metaverse space providing unit 100 may provide a metaverse space for a cognitive and social treatment service to a device of a child with developmental disabilities and a device of a therapist.

Herein, the metaverse space may include an activity space where the device of the child with developmental disabilities performs a mission of the device of the therapist and an observation space where an activity of the device of the child with developmental disabilities is evaluated and fed back. Herein, the activity space may include a social interaction space (e.g., a school playground, etc.) to achieve the purpose of treating the child with developmental disabilities.

For example, referring to FIG. 2, when the device of the child with developmental disabilities enters a metaverse space 20, the device of the child with developmental disabilities may check, through a noticeboard 201, information about a treatment activity in which the child with developmental disabilities is to participate.

The metaverse space 20 may provide a chat interface where the device of the child with developmental disabilities can chat with other devices (e.g., a device of another child with developmental disabilities who participates in the metaverse space, the device of the therapist, etc.). In this case, a chat content 203 of the device of the child with developmental disabilities and the other devices may be displayed on the metaverse space 20.

An image 205 of a facial expression and an upper body movement of the child with developmental disabilities captured by a camera may be displayed on the metaverse space 20.

The device of the child with developmental disabilities may participate in the cognitive and social treatment service through a child character 207 mapped to the device of the child with developmental disabilities according to a pre-created treatment program. Herein, the cognitive and social treatment service is conducted based on the pre-created treatment program.

In the metaverse space 20, an emoticon interface for expressing emotions of the child with developmental disabilities may be provided to the device of the child with developmental disabilities. Herein, emoticons used by the device of the child with developmental disabilities may be used to analyze the emotions of the child with developmental disabilities.

In the metaverse space 20, a therapist-dedicated interface 209 may be provided to the device of the therapist to enhance control of the device of the therapist during treatment of the child with developmental disabilities.

Referring back to FIG. 1, the data collection unit 110 may collect life-logging data including user data and cognitive and social response data of the child with developmental disabilities who is being diagnosed or treated in the metaverse space.

Herein, the user data may include image data including a facial expression and an upper body movement of the child with developmental disabilities and voice data of the child with developmental disabilities.

The cognitive and social response data may include activity information of the child character controlled by the device of the child with developmental disabilities in the metaverse space. Herein, the activity information of the child character may be derived based on shift coordinates of the child character moving to the activity space for the treatment in the metaverse space. The activity of the child character may be indexed depending on the size of a movement of the child character which is calculated based on the shift coordinates of the child character.

The data collection unit 110 may collect life-logging data which further includes biometric data collected from a wearable device worn by the child with developmental disabilities during the treatment.

Herein, the wearable device is used to measure a non-verbal reaction of the child with developmental disabilities, and may include, for example, a wearable device having a body attachment type or a shirt type. The wearable device may be configured to sense and process bio-signals of the child with developmental disabilities.

Herein, the biometric data collected from the wearable device may include, for example, heart rate (HR) information, heart rate variability (HRV) information, sleep pattern information (e.g., a sleep length and a sleep quality), respiratory rate information, stress index information, and amount and size information of physical activity of the child with developmental disabilities.

The mental state estimation unit 120 may estimate a mental state of the child with developmental disabilities from the life-logging data through an artificial intelligence model.

For example, the mental state estimation unit 120 may derive the mental state of the child with developmental disabilities analyzed from the life-logging data as at least one of pre-defined categories of mental states (e.g., happy, angry, unpleasant, afraid, sad, surprised, and neutral (insensitive)).

The mental state estimation unit 120 may transmit the estimated mental state of the child with developmental disabilities through the artificial intelligence model to the device of the therapist in a regular periodic manner before and during a treatment session of the child with developmental disabilities. In this case, the estimated mental state of the child with developmental disabilities through the artificial intelligence model may be used as an auxiliary tool by the therapist during cognitive and social treatment of the child with developmental disabilities, and may also be used for a next treatment session after the end of the treatment session.

The therapist may determine a social treatment plan for the child with developmental disabilities, an involvement method and an intensity based on an information value of the mental state of the child with developmental disabilities and an objective index professionally determined by the therapist.

The mental state estimation unit 120 may extract facial features, such as eyes, nose and mouth, of the child with developmental disabilities from image data associated with utterance information of the therapist among the image data, and may estimate a first mental state of the child with developmental disabilities based on muscle activity caused by changes in distance between the extracted facial features. Herein, the utterance information of the therapist may include, for example, utterances for reinforcement and encouragement (compliment, action control, etc.).

The mental state estimation unit 120 may extract coordinate values of the facial features of the child with developmental disabilities from the image data of the child with developmental disabilities, evaluate facial muscle activity of the child with developmental disabilities based on the extracted ordinate values of the facial features, and estimate the first mental state for determining an emotional reaction of the child with developmental disabilities based on the facial muscle activity of the child with developmental disabilities.

Herein, the first mental state is used to check a response of the child with developmental disabilities to the treatment and a treatment effect, and may be classified into one of general categories, such as negative, positive, and neutral, and may also be classified into one of detailed emotion categories, such as happy, sad, and surprised.

The mental state estimation unit 120 may convert voice data of the child with developmental disabilities recorded during the treatment of the child with developmental disabilities into text by text mining, and may estimate a second mental state for determining an utterance emotion of the child with developmental disabilities by analyzing emotions in the converted text.

The mental state estimation unit 120 may estimate the second mental state of the child with developmental disabilities from voice data of the child with developmental disabilities associated with the utterance information of the therapist among the voice data.

The mental state estimation unit 120 may estimate the second mental state of the child with developmental disabilities from a voice tone and a response content of the child with developmental disabilities in consideration of an utterance time for which the therapist tries to make utterances for reinforcement and encouragement, and an utterance content.

Herein, an utterance of the therapist for reinforcement or encouragement in a specific situation may serve as a marker for determining whether the child with developmental disabilities has shown a socially appropriate behavior.

The image data and voice data of the child with developmental disabilities associated with the utterance information of the therapist including reinforcement and encouragement may serve as key data to increase the accuracy in estimating a mental state (emotional state) of the child with developmental disabilities.

The mental state estimation unit 120 may calculate a biometric variance between a daily activity and an activity during the treatment of the child with developmental disabilities based on the biometric data of the child with developmental disabilities, and may estimate a third mental state of the child with developmental disabilities based on the calculated biometric variance.

For example, the mental state estimation unit 120 may input the biometric variance into a pre-trained classification model to classify the mental state of the child with developmental disabilities into one of representative emotion categories (negative emotion, positive emotion, and neutral emotion) or into one of detailed emotion categories (happy, angry, unpleasant, afraid, sad, surprised, insensitive, etc.).

The mental state estimation unit 120 may estimate an average activity state of the child with developmental disabilities based on the biometric data and cognitive and social response data of the child with developmental disabilities.

The treatment service providing unit 130 may provide the cognitive and social treatment service of the child with developmental disabilities to the device of the child with developmental disabilities based on the estimated mental state of the child with developmental disabilities.

As for an existing offline social treatment program, a therapist in charge monitors and evaluates children with developmental disabilities for a long time and then groups children with similar tendencies or complementary tendencies to conduct a group mediation. In this case, children with appropriate conditions, such as age, sex, and tendency, are searched for from a limited candidate pool and matched to each other and children as group members in the same group need to visit a physical space regularly at the same hour. Therefore, the children often have to wait several months or longer.

The present disclosure can overcome the conventional time and space limitations, secure a participant pool composed of a greater number of children, and provide a flexible mediation schedule.

Specifically, the treatment service providing unit 130 may group children with developmental disabilities who have similar social and cognitive skills to those of the child with developmental disabilities among a plurality of children with developmental disabilities who participates in the metaverse space, and may provide information about the group to the device of the therapist.

The mental state estimation unit 120 may estimate an average activity state of each child with developmental disabilities based on biometric data and cognitive and social response data (amount of activity in the metaverse space) of each of the plurality of children with developmental disabilities, and may categorize an emotion of each child with developmental disabilities based on voice data of each child with developmental disabilities and chat contents in the metaverse space.

The treatment service providing unit 130 may derive an emotional valence of each child with developmental disabilities based on the average activity state and emotion category of each child with developmental disabilities, and may form a treatment group by grouping children with developmental disabilities who have similar social and cognitive skills based on the derived emotional valence of each child with developmental disabilities.

The treatment service providing unit 130 may form a treatment group for cognitive and social treatment based on prior information (e.g., diagnosis name, pathologic level, psychological test information about each child, etc.) collected for each child with developmental disabilities.

The treatment service providing unit 130 may regroup the children with developmental disabilities based on a reinforcement point score acquired by each child with developmental disabilities in each initial treatment group during a first treatment session and a frequency (number of times of score acquisition, etc.), and may reset a treatment group for a next treatment session. Also, the treatment service providing unit 130 may provide information about the regrouped treatment group to the device of the therapist.

The treatment service providing unit 130 may give an activity score to the child with developmental disabilities based on the user data and cognitive and social response data of the child with developmental disabilities who is being treated according to the cognitive and social treatment service.

For example, the treatment service providing unit 130 may give an activity score to the child with developmental disabilities depending on whether the user data (image data and voice data) and cognitive and social response data of the child with developmental disabilities correspond to an appropriate behavior intended in a program of the cognitive and social treatment service. Herein, the appropriate behavior refers to a behavior following the instructions of the therapist, and may include adaptive behaviors, such as making an utterance as expected by the therapist, performing a task of a treatment class, and moving to a predetermined treatment space. Meanwhile, an inappropriate behavior may include non-adaptive behaviors, such as not following the instructions, using banned words, screaming, self-torturing, and escaping from a treatment space.

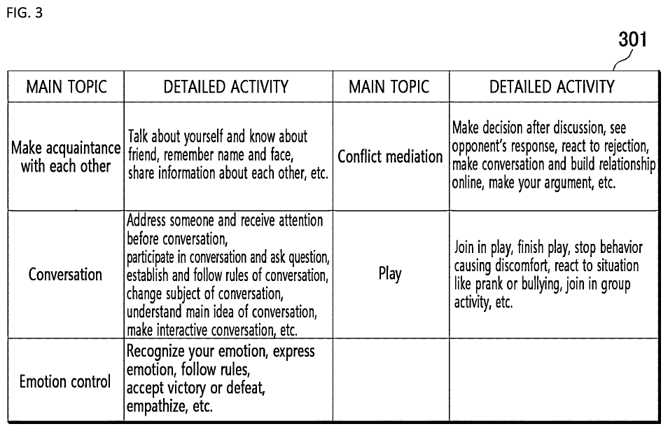

For example, referring to FIG. 3, the treatment service providing unit 130 may give an activity score to a treatment group including the child with developmental disabilities based on a treatment goal according to a treatment scenario topic 301 and an evaluation criterion, and may give an activity score to each child with developmental disabilities.

The treatment scenario topic 301 may include, for example, conversation skills, compromise and conflict resolution skills, play-related skills, and emotion recognition and control-related skills included in general social skill training.

The treatment service providing unit 130 may establish and manage a treatment goal and an evaluation criterion for each treatment group or each child with developmental disabilities. Herein, the treatment goal and the evaluation criterion may be uniformly applied to the entire treatment group or differently applied to each child.

For example, as for the treatment goal and the evaluation criterion, the treatment service providing unit 130 may use a criterion suitable for a topic of a social skill session and a detailed activity (e.g., topic: conversation, detailed activity: addressing someone and receiving attention before conversation), or may establish and manage a goal for resolving the problem each child has (e.g., a decrease in repeated head hitting and a decrease in frequency of screaming) in addition to the treatment goal.

The overall treatment goal and the treatment goal for each child may be independently applied for the entire session, and the treatment goal for each child may be established based on objective information acquired in advance by the therapist through a clinical evaluation or diagnosis before participation in the treatment program.

The treatment service providing unit 130 may evaluate a performance score of the child with developmental disabilities and a treatment effect for each treatment session based on the user data and cognitive and social response data of the child with developmental disabilities who is being treated according to the cognitive and social treatment service.

The treatment service providing unit 130 may evaluate the change and improvement of the child with developmental disabilities based on an evaluation result of the child with developmental disabilities from each treatment session and cumulative treatment sessions, and may evaluate the child with developmental disabilities according to a plurality of evaluation indexes.

When a treatment session for the child with developmental disabilities does not proceed under predetermined conditions, the treatment service providing unit 130 may control the child character of the device of the child with developmental disabilities to automatically execute a program satisfying the predetermined conditions.

For example, when the child with developmental disabilities does not move to a specific space (e.g., playground, school, etc.) where a treatment session proceeds within a predetermined time limit set by the therapist, the treatment service providing unit 130 may immediately move the child character of the device of the child with developmental disabilities to the specific space.

To suppress an influence of voice and facial expression of another child with developmental disabilities on treatment of the child with developmental disabilities, the treatment service providing unit 130 may set a boundary line to distinguish communication between the children with developmental disabilities in the metaverse space, and may display a boundary region by using a spotlight function.

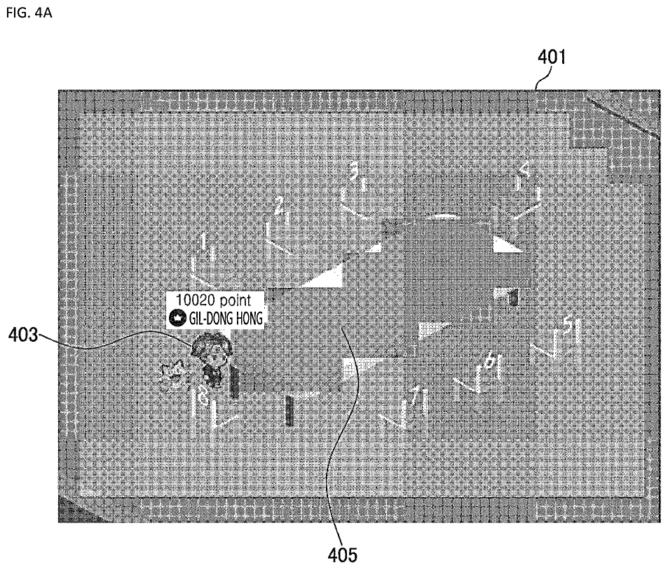

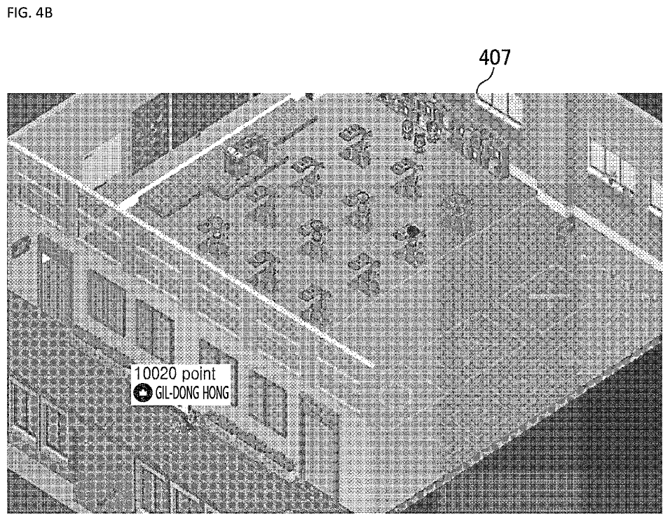

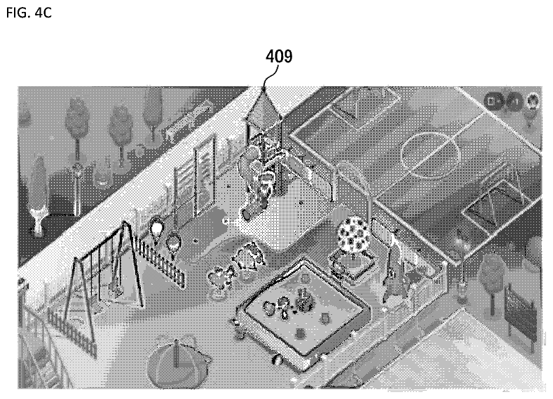

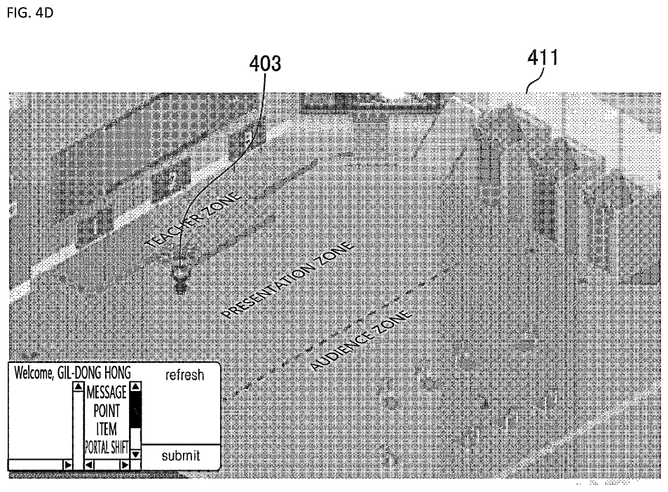

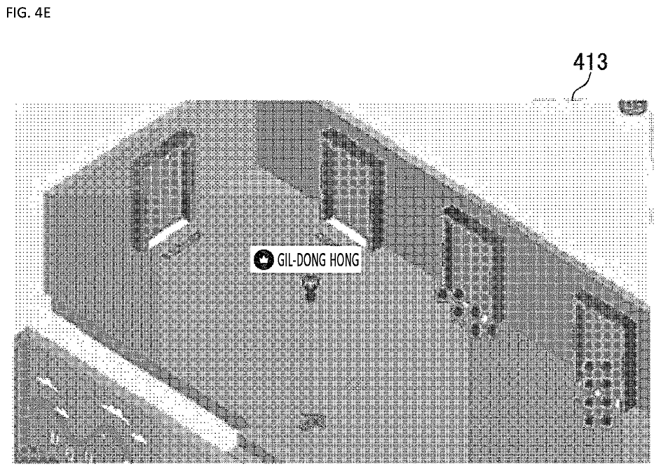

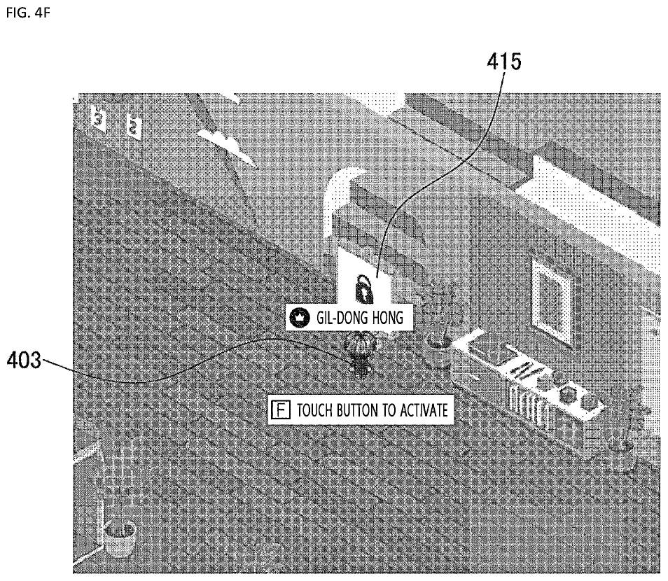

For example, referring to FIG. 4A, when a child character 403 of the device of the child with developmental disabilities moves to a specific space 401, devices of children with developmental disabilities located in a shaded region 405 can share their voices and images, but the device of the child with developmental disabilities located outside the shaded region cannot check the voice and image of another child with developmental disabilities located in the shaded region.

The treatment service providing unit 130 may provide, to the device of the child with developmental disabilities, a treatment space suitable for social interaction in the age group of the child with developmental disabilities.

For example, referring to FIG. 4B, if the child with developmental disabilities is in the low-age group, the treatment service providing unit 130 may use a fixed character to reenact a situation which can occur in a classroom 407, and may provide an interaction which the child with developmental disabilities can learn from the situation. Also, the treatment service providing unit 130 may provide, to the device of the child with developmental disabilities, a character motion with which the child with developmental disabilities needs to be trained in a social bullying situation. Further, the treatment service providing unit 130 may provide, to the device of the child with developmental disabilities, a space where the child with developmental disabilities can watch and evaluate the activities of others by means of role play.

For example, referring to FIG. 4C, if the child with developmental disabilities is in the low-age group, the treatment service providing unit 130 may use a fixed character to reenact a situation which can occur in a playground 409, and may provide, to the device of the child with developmental disabilities, a character motion which the child with developmental disabilities needs to learn from the situation.

For example, referring to FIG. 4D, if the child with developmental disabilities is in the high-age group, the treatment service providing unit 130 may provide an activity space 411 where the child character 403 of the device of the child with developmental disabilities may make a presentation or conduct a team activity, and may provide, through a noticeboard interface (e.g., a white board, etc.), a task which the child with developmental disabilities needs to perform. Herein, the activity space 411 is equipped with a meeting room and a locker room for each team for team activities essential to social treatment. When the child character of the device of the child with developmental disabilities enters the locker room, the color of clothes of the child character is changed to the color of clothes of a team to which the child character belongs in order to distinguish the team.

For example, referring to FIG. 4E, the treatment service providing unit 130 may provide a game zone 413 for developing teamwork between the children with developmental disabilities and arousing interest of the children with developmental disabilities. Herein, the game zone 413 may be composed of a group game zone where congenial children can enjoy a game, an individual game zone where an individual can enjoy a game, a mini room where the child with developmental disabilities can decorate his/her own space, and a game space map through which the child character can move to various game spaces (e.g., Spot the Difference, Room Escape Game, etc.).

Herein, the mini room is a space created in a metaverse space for reinforcement and encouragement which are key mechanisms of the cognitive and social treatment, and is a space which can be decorated by the child with developmental disabilities as his/her own space with preferred items purchased with his/her points.

The game zone 413 is preferred particularly by children and thus needs to be controlled by the therapist, and may be designed to allow entrance of the children only when approval of the therapist is completed.

Since a free movement of the child with developmental disabilities between spaces needs to be banned for the purpose of treatment, the treatment service providing unit 130 may set a boundary line that distinguishes between spaces to allow a movement upon approval of the therapist.

For example, referring to FIG. 4F, when a password of the therapist is input to a cloak 415 set up on the boundary line that distinguishes between spaces, the treatment service providing unit 130 may clear the cloak 415 and allow the child character 403 of the child with developmental disabilities to enter another space.

When an activity of an observer (e.g., parents, assistant therapist, etc.) is needed for the child with developmental disabilities, the treatment service providing unit 130 may allow a separate observer character to participate in the treatment of the child with developmental disabilities. Herein, the separate observer character is hidden in the treatment program and may be designed to respond according to a predetermined criterion (e.g., saying “hello” when someone comes up, etc.).

Also, the treatment service providing unit 130 may share, with a device of the observer, a view of the therapist (i.e., a view that facilitates observation of a process of treatment of the child with developmental disabilities). Accordingly, the observers can directly observe the process of treatment of the child with developmental disabilities.

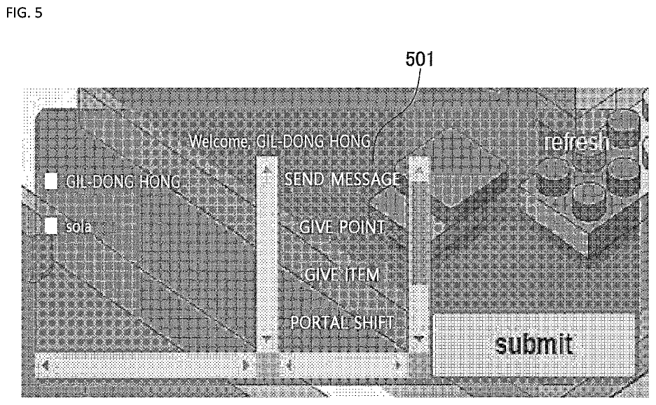

The therapist function providing unit 140 may provide a therapist-dedicated interface to the device of the therapist that needs to be involved in the treatment of the child with developmental disabilities. Herein, the therapist-dedicated interface may include therapist-dedicated functions designed for the purpose of cognitive and social treatment in the metaverse. For example, referring to FIG. 5, a therapist-dedicated interface 501 may include at least one of a function of sending a message to the device of the child with developmental disabilities, a function of giving a point, a function of giving an item, a portal shift function, a function of inputting a team score, a function of setting a timer, a function of setting a motion in a cyberbullying situation, a function of writing a treatment result report, a function of facilitating observation, and a function of setting a password.

The therapist may select a name of each child with developmental disabilities or a treatment group from the therapist-dedicated interface 501 and apply at least one of the plurality of therapist-dedicated functions, and may perform a refresh function to reset an input value.

The function of sending a message of the therapist-dedicated interface 501 is designed to complement the problem of the child with developmental disabilities with concentration on interactions with others and social situations and enable the child with developmental disabilities to concentrate on the treatment. The therapist may provide each instruction to the device of the child with developmental disabilities by performing a one-to-one message function. When the device of the therapist sends a one-to-one message, the message is displayed on a screen of the device of the child with developmental disabilities and thus can be immediately checked by the child with developmental disabilities.

The function of giving a point of the therapist-dedicated interface 501 is designed to give or deduct a point to or from the device of the child with developmental disabilities depending on a response of the child with developmental disabilities (or children belonging to the treatment group). This function corresponds to reinforcement which is a key mechanism of the cognitive and social treatment.

When a point is given or deducted by performing the function of giving a point, a character name and a point score of the child with developmental disabilities to or from whom a point has been given or deducted are displayed in the metaverse space in real time and thus can be seen by all of the members being treated.

Also, the given or deducted point score is automatically recorded and monitored by the treatment service mediation server 10, and may be used to determine a social skill of the child with developmental disabilities depending on the time at which the point score is provided. Before and after the point score is provided, a change in facial expression of the child with developmental disabilities and variance in biometric data may be tracked, and the tracked information may be used to verify the accuracy and reliability of the prepared treatment program.

The point given to the child with developmental disabilities may serve as social currency to purchase a pet or item preferred by the child with developmental disabilities. The pet purchased by the device of the child with developmental disabilities may be designed to follow the child character of the device of the child with developmental disabilities.

Further, the point may be used by the device of the child with developmental disabilities to purchase a preferred item for decorating the mini room where the child with developmental disabilities can decorate his/her own space, and the device of the child with developmental disabilities may arrange the item on his/her own.

The function of giving an item of the therapist-dedicated interface 501 is designed to directly provide an item to the child in addition to the point. With this function, it is possible to give the child with developmental disabilities a motivation (reinforcer) which is stronger than collecting the point by child with developmental disabilities.

The portal shift function of the therapist-dedicated interface 501 is designed to suppress an escape of the child character of the child with developmental disabilities from the treatment space. With this function, the therapist can force the child character to move to a space designated by the therapist.

The function of inputting a team score of the therapist-dedicated interface 501 is designed to input a score for a result of team treatment activity by the therapist during the cognitive and social treatment requiring a group activity.

The function of setting a timer of the therapist-dedicated interface 501 is designed to put the child with developmental disabilities to achieve a treatment goal within a limited time period. For example, when the device of the therapist performs the function of setting a timer to set a certain time limit, the timer is located at the top-left of the screen of the treatment space to count down. Accordingly, the child with developmental disabilities can perform a treatment activity while visually checking the time limit.

The function of setting a motion in a cyberbullying situation of the therapist-dedicated interface 501 is designed to provide the metaverse space with activities (training against bullying), such as cybercrime or online sexual harassment, which cannot be implemented offline for safety reasons.

For example, a situation may be simulated in which child characters of the devices of children with developmental disabilities fall over to get hurt by mutual attacks. Accordingly, a scenario for a situation where the child character of the child with developmental disabilities serves as an attacker and directly attacks the child character of another child with developmental disabilities, a scenario for a situation where the child character serves as a victim and is directly attacked by the child character of another child with developmental disabilities, or a scenario for a situation where the child character of the child with developmental disabilities attacks a teacher or a regular adult instead of peers may be created to provide a treatment program by which the child with developmental disabilities can be trained to control aggression and show an appropriate social response.

This function may be applied to a part of the entire treatment session (e.g., a first session). Frequent activation of this function may cause the child with developmental disabilities to cultivate violence or defer participation in the treatment during the entire treatment session. Therefore, the therapist can set a time period in which cyberbullying (falling over) is activated after selecting an account of the child with developmental disabilities and thus control the child with developmental disabilities.

The function of writing a treatment result report of the therapist-dedicated interface 501 is designed for the therapist to write, after the treatment, a treatment result report on changes in emotions and social skills of each child during the treatment based on information and biometric information of the child with developmental disabilities acquired by the therapist during the entire treatment session. Herein, the treatment result report may include the overall emotion ratio of the child with developmental disabilities according to the treatment program and information about physical activities (e.g., heart rate), and may also include information for checking changes before and after the therapist provides main reinforcement (e.g., compliment and point). The treatment result report is written after the treatment of the child with developmental disabilities, and may be used by the therapist as reference materials to form a group of children for a next treatment session, check cognitive and social skills of each child, or establish a personalized treatment goal.

The function of facilitating observation of the therapist-dedicated interface 501 is designed for the therapist to share a view of the therapist with an observer of the child with developmental disabilities.

The function of setting a password of the therapist-dedicated interface 501 is designed to set the therapist's approval, such as a password, to control a movement of the child character of the child with developmental disabilities to a specific space.

After the treatment of the child with developmental disabilities is finished, the treatment service providing unit 130 may provide, to a device of parents, a trial service which enables the parents to simulate the treatment activity of the child with developmental disabilities.

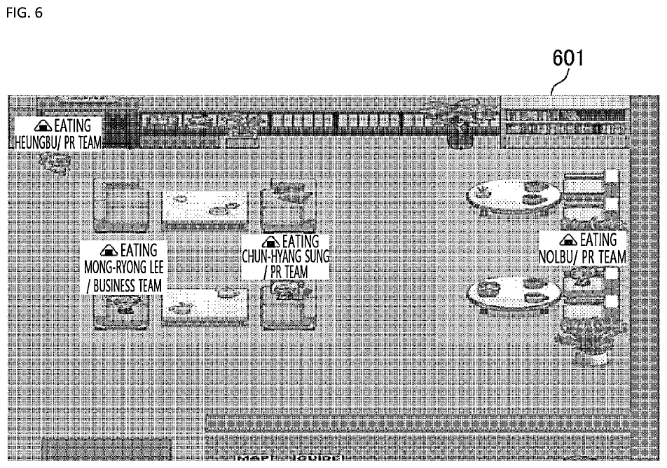

For example, referring to FIG. 6, the treatment service providing unit 130 may provide, to the device of the parents, a parental metaverse space 601 in which the parents can simulate the treatment activity of the child with developmental disabilities.

The treatment result providing unit 150 may provide the treatment result report on the cognitive and social treatment service to the device of the parents of the child with developmental disabilities. Herein, the treatment result report may include at least one of an access time for which the device of the child with developmental disabilities accesses the metaverse space during the treatment session, an utterance time of the child with developmental disabilities, total points acquired by the child character controlled by the child with developmental disabilities, activity information (e.g., items for decorating the pet or the mini room, a frequency of falling over of the child character, etc.), and information about emotions and social skills of the child with developmental disabilities.

Further, the treatment result report may include evaluation index improvement and biometric changes (e.g., heart rate, stress index, etc.) of the child with developmental disabilities measured by the therapist before and after the treatment of the child with developmental disabilities, and suggested activities for the child with developmental disabilities after the treatment.

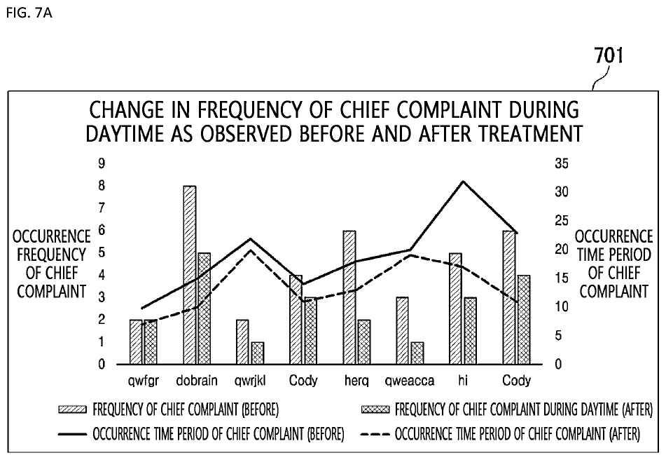

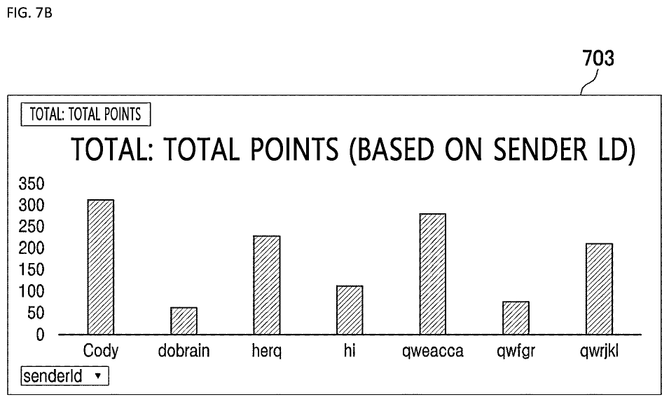

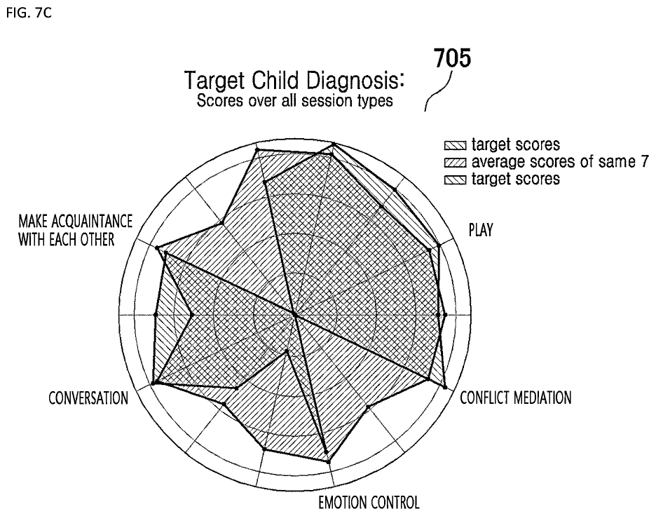

For example, referring to FIG. 7A, the treatment service providing unit 130 may provide, to the device of the parents of the child with developmental disabilities, a treatment result report including a graph 701 of occurrence frequency and occurrence time period of chief complaint (e.g., psychological problems such as depression and anxiety and cognitive and emotional problems including specific asocial activities) of the child with developmental disabilities, and explanation thereof. The graph 701 shows how chief complaint of the child with developmental disabilities has changed before and after the treatment.

For example, referring to FIG. 7B, the treatment service providing unit 130 may provide, to the device of the parents of the child with developmental disabilities, a bar graph 703 of cumulative points of a plurality of children with developmental disabilities, and explanation thereof through the treatment result report. The bar graph 703 can be used to compare the child with developmental disabilities to another child with developmental disabilities in terms of treatment activity performance.

For example, referring to FIG. 7C, the treatment service providing unit 130 may provide, to the device of the parents of the child with developmental disabilities, a radial chart 705 and explanation thereof through the treatment result report. The radial chart 705 shows effects of the social treatment on the child with developmental disabilities for respective evaluation indexes (making acquaintance with each other, conversation, emotion control, conflict mediation, and play).

The treatment service providing unit 130 may create an abstract by extracting a primary situation and a scene in which the child with developmental disabilities participates during the treatment session, and may provide the device of the parents with the abstract

The treatment service providing unit 130 may provide the device of the parents with log information that is listed containing changes in activities of the child with developmental disabilities and changes in evaluation indexes over time or focusing on significant events.

Meanwhile, it would be understood by a person with ordinary skill in the art that each of the metaverse space providing unit 100, the data collection unit 110, the mental state estimation unit 120, the treatment service providing unit 130, the therapist function providing unit 140, and the treatment result providing unit 150 can be implemented separately or in combination with one another.

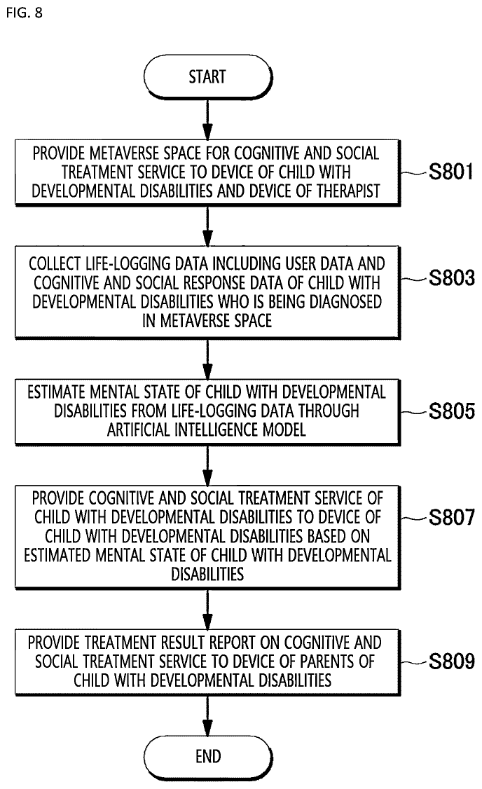

FIG. 8 is a flowchart showing a method of providing a cognitive and social treatment service of the child with developmental disabilities according to an embodiment of the present disclosure.

Referring to FIG. 8, in a process S801, the treatment service mediation server 10 may provide a metaverse space for a cognitive and social treatment service to a device of a child with developmental disabilities and a device of a therapist.

In a process S803, the treatment service mediation server 10 may collect life-logging data including user data and cognitive and social response data of the child with developmental disabilities who is being diagnosed in the metaverse space.

In a process S805, the treatment service mediation server 10 may estimate a mental state of the child with developmental disabilities from the life-logging data through an artificial intelligence model.

In a process S807, the treatment service mediation server 10 may provide the cognitive and social treatment service of the child with developmental disabilities to the device of the child with developmental disabilities based on the estimated mental state of the child with developmental disabilities.

In a process S809, the treatment service mediation server 10 may provide a treatment result report on the cognitive and social treatment service to a device of parents of the child with developmental disabilities.

In the descriptions above, the processes S801 to S809 may be divided into additional processes or combined into fewer processes depending on an embodiment. In addition, some of the processes may be omitted and the sequence of the processes may be changed if necessary.

The embodiment of the present disclosure can be embodied in a storage medium including instruction codes executable by a computer such as a program module executed by the computer. A computer-readable medium can be any usable medium which can be accessed by the computer and includes all volatile/non-volatile and removable/non-removable media. Further, the computer-readable medium may include all computer storage media. The computer storage media include all volatile/non-volatile and removable/non-removable media embodied by a certain method or technology for storing information such as computer-readable instruction code, a data structure, a program module or other data.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A treatment service mediation server that provides a cognitive and social treatment service of a child with developmental disabilities, comprising:
a metaverse space providing unit configured to provide a metaverse space for the cognitive and social treatment service to a device of the child with developmental disabilities and a device of a therapist;
a data collection unit configured to collect life-logging data including user data and cognitive and social response data of the child with developmental disabilities during a diagnostic process in the metaverse space;
a mental state estimation unit configured to utilize an artificial intelligence model to estimate a mental state of the child with developmental disabilities based on the collected life-logging data;
a treatment service providing unit configured to provide the cognitive and social treatment service of the child with developmental disabilities to the device of the child based on the estimated mental state; and
a treatment result providing unit configured to provide a treatment result report on the cognitive and social treatment service to a device of parents of the child.

2. The treatment service mediation server of claim 1,
wherein the user data includes image data including a facial expression and an upper body movement of the child with developmental disabilities and voice data of the child with developmental disabilities, and
the cognitive and social response data includes activity information of a child character controlled by the device of the child with developmental disabilities in the metaverse space.

3. The treatment service mediation server of claim 1,
wherein the life-logging data further includes biometric data collected from a wearable device worn by the child with developmental disabilities during treatment.

4. The treatment service mediation server of claim 2,
wherein the mental state estimation unit is further configured to extract facial features of the child with developmental disabilities from image data associated with utterance information of the therapist among the image data, and estimate a first mental state of the child with developmental disabilities based on muscle activity caused by changes in distance between the extracted facial features.

5. The treatment service mediation server of claim 4,
wherein the mental state estimation unit is further configured to estimate a second mental state of the child with developmental disabilities from voice data of the child with developmental disabilities associated with the utterance information of the therapist among the voice data.

6. The treatment service mediation server of claim 3,
wherein the mental state estimation unit is further configured to calculate a biometric variance between a daily activity and an activity during the treatment of the child with developmental disabilities based on the biometric data, and estimate a third mental state of the child with developmental disabilities based on the calculated biometric variance.

7. The treatment service mediation server of claim 6,
wherein the mental state estimation unit is further configured to estimate an average activity state of the child with developmental disabilities based on the biometric data and the cognitive and social response data of the child with developmental disabilities.

8. The treatment service mediation server of claim 1,
wherein the treatment service providing unit is further configured to group children with developmental disabilities who have similar social and cognitive skills to those of the child with developmental disabilities among a plurality of children with developmental disabilities who participates in the metaverse space, and provide information about the group to the device of the therapist.

9. The treatment service mediation server of claim 1,
wherein the treatment service providing unit is further configured to evaluate a performance score and a treatment effect of the child with developmental disabilities based on the user data and cognitive and social response data of the child with developmental disabilities who is being treated according to the cognitive and social treatment service.

10. The treatment service mediation server of claim 1,
wherein when a treatment session for the child with developmental disabilities does not proceed under predetermined conditions, the treatment service providing unit is further configured to control a child character of the device of the child with developmental disabilities to automatically execute a program satisfying the predetermined conditions.

11. The treatment service mediation server of claim 1,
wherein the metaverse space includes an activity space where the device of the child with developmental disabilities performs a mission of the device of the therapist and an observation space where an activity of the device of the child with developmental disabilities is evaluated and fed back.

12. The treatment service mediation server of claim 1, further comprising:
a therapist function providing unit configured to provide a therapist-dedicated interface to the device of the therapist that needs to be involved in the treatment of the child with developmental disabilities.

13. The treatment service mediation server of claim 12,
wherein the therapist-dedicated interface includes at least one of a function of sending a message to the device of the child with developmental disabilities, a function of giving a point, a function of giving an item, a portal shift function, a function of inputting a team score, a function of setting a timer, and a function of setting a motion in a cyberbullying situation.

14. The treatment service mediation server of claim 1,
wherein the treatment result report includes at least one of an access time for which the device of the child with developmental disabilities accesses the metaverse space during a treatment session, an utterance time of the child with developmental disabilities, total points acquired by a child character controlled by the child with developmental disabilities, activity information, and information about emotions and social skills of the child with developmental disabilities.

15. The treatment service mediation server of claim 1,
wherein after the treatment of the child with developmental disabilities is finished, the treatment service providing unit is further configured to provide, to the device of the parents, a trial service which enables the parents to simulate a treatment activity of the child with developmental disabilities.

16. A treatment service mediation method that provides a cognitive and social treatment service of a child with developmental disabilities and is performed by a treatment service mediation server, comprising:
providing a metaverse space for the cognitive and social treatment service to a device of the child with developmental disabilities and a device of a therapist;
collecting life-logging data including user data and cognitive and social response data of the child with developmental disabilities during a diagnostic process in the metaverse space;
utilizing an artificial intelligence model to estimate a mental state of the child with developmental disabilities based on the collected life-logging data;
providing the cognitive and social treatment service of the child with developmental disabilities to the device of the child based on the estimated mental state of the child; and
providing a treatment result report on the cognitive and social treatment service to a device of parents of the child.

17. A non-transitory computer-readable medium storing a computer program including a sequence of instructions for providing a cognitive and social treatment service of a child with developmental disabilities, which when executed by a computing device, cause a computing device to:
provide a metaverse space for the cognitive and social treatment service to a device of the child with developmental disabilities and a device of a therapist;
collect life-logging data including user data and cognitive and social response data of the child with developmental disabilities during a diagnostic process in the metaverse space;
utilize an artificial intelligence model to estimate a mental state of the child with developmental disabilities based on the collected life-logging data;
provide the cognitive and social treatment service of the child with developmental disabilities to the device of the child based on the estimated mental state of the child; and
provide a treatment result report on the cognitive and social treatment service to a device of parents of the child.
